(19) United States
(12) Patent Application Publication
(10) Pub. No.: US 2018/0117196 A1
(43) Pub. Date: May 3, 2018

(54) PLASMA SYSTEMS DRIVEN BY DC VOLTAGE AND METHODS OF USING THE SAME

(71) Applicant: Purdue Research Foundation, West Lafayette, IN (US)

(72) Inventors: Alexey Shashurin, West Lafayette, IN (US); Xingxing Wang, West Lafayette, IN (US)

(73) Assignee: Purdue Research Foundation, West Lafayette, IN (US)

(21) Appl. No.: 15/729,971

(22) Filed: Oct. 11, 2017

Related U.S. Application Data
(60) Provisional application No. 62/407,907, filed on Oct. 13, 2016.

(51) Int. Cl. A61L 2/14; A61L 2/26; A23B 7/144; A23L 3/3409

(52) U.S. Cl. CPC A61L 2/14; A61L 2/26; A23B 7/144; A23V 2002/00; A61L 2202/11; A61L 2202/24; A23L 3/3409

(57) ABSTRACT

A plasma generating system capable of generating a cold plasma. The plasma generating includes two electrodes, a DC voltage source capable of applying a constant DC voltage between the two electrodes, an insulator located in proximity of the two electrodes, and a gas filling a gap between the two electrodes, wherein cold plasma in the form of series of repetitive streamer breakdowns of the gas is generated, in response to the constant DC voltage applied between the two electrodes. A method of producing and storing a sterilizing gas. The method includes providing a flow of a gas into a chamber, generating cold plasma through repetitive streamer breakdowns of the gas in response to an applied DC voltage, resulting in the gas becoming a sterilizing gas. A method of sterilizing an object. The method includes exposing an object to be sterilized to the sterilizing gas for a period of time.

### CROSS-REFERENCE TO RELATED APPLICATIONS

The present patent application is related to and claims the priority benefit of U.S. Provisional Patent Application Ser. No. 62/407,907 filed Oct. 13, 2016 the contents of which are incorporated in their entirety herein by reference.

### STATEMENT REGARDING GOVERNMENT FUNDING

This invention was made with government support under Award No. PHY1465061 awarded by the National Science Foundation. The government has certain rights in the invention.

### TECHNICAL FIELD

This disclosure generally relates to cold plasma, especially those driven by Direct Current (DC) voltage.

### BACKGROUND

This section introduces aspects that may help facilitate a better understanding of the disclosure. Accordingly, these statements are to be read in this light and are not to be understood as admissions about what is or is not prior art.

Cold plasmas are widely used nowadays in the fields of bio-engineering, medicine, food processing etc. Generally, term cold plasma refers to specific type of ionized gas (plasma) when gas is cold, namely its temperature is typically close to room temperature, but can vary in the general range of 300-400 K Therefore, cold plasmas operate under the threshold of thermal damage to the tissue (eliminating tissue burn) and induce specific chemical responses on the cellular level and can offer minimum invasive surgery technique. Currently, cold plasmas find wide application in the areas of sterilization and disinfection. Cold plasmas effectively kill bacteria, while harmless thermally since operates at nearly room temperature. More exotic utilization includes cancer treatment including lung, bladder, skin, head and neck, brain, pancreatic tumors etc., skin dentistry, drug delivery, dermatology, cosmetics, wound healing, cellular modifications, etc.

Conventional cold plasmas are excited in helium (He) flow exhausted from the discharge tube into open air. Multiple parameters of He plasma jets were measured previously including plasma density, temperatures of various species, electrical currents etc. Typically, plasma electron density nₑ is in the range of 10¹²-10¹³ cm⁻³ while the temperature of heavy species is near the room temperature at 300-350 K.

Conventional cold plasmas are excited using AC or pulsed DC power supplies operating in kV range and frequencies around 10s of kHz. In those cases, breakdown takes place once every cycle of the applied high voltage (HV) when the voltage applied to the electrode reaches the breakdown threshold. The breakdown is associated with development of streamer tip propagating at characteristic velocities in the range of 10⁶-10⁸ cm/s increasing with the high voltage magnitude. The duration of each individual streamer does not exceed the period of time of several μs and stops where the presence of the oxygen in the He jet increases along the jet to about 1 percent. The plasma remaining in the streamer channel decays shortly afterwards (on time scale of about several μs). The next breakdown event occurs on the next cycle of the applied AC high voltage or with the application of the next high voltage pulse. Thus, the repetition frequency of the discharge is fully governed by the discharge driving power supply operation frequency.

Traditionally cold plasmas are driven by AC or pulsed-DC HV operating in the kV range, but such power sources have certain disadvantages. Firstly, the time-varying power sources of the system are often associated with high cost, especially if a wide range of operating frequency is required. Another downside of the application of AC/pulsed-DC HV is that there are serious safety concerns with their operation. Operation at AC and/or pulsed-DC high voltages reduces resistance of insulating shields due to capability of displacement currents conduction. This increases danger of electric breakdown between the parts of the system and raises possibility of electric shocks for the personnel operating the equipment at touching insulated high voltage lines and other surrounding objects. These safety concerns tremendously increase requirements to the insulation shields and make use of these devices impractical in many cases especially if complicated systems with large number of sensitive components are employed. Thirdly, operating the time-varying HV sources causes EMI with other sensitive electronics nearby that can potentially be disastrous during some medical procedures.

Thus there exists an unmet need for a system capable of generating a cold plasmas without the problems of EMI and leakage currents.

### SUMMARY

A plasma generating system capable of generating a cold plasma is disclosed. The plasma generating includes two electrodes, wherein one of the two electrodes is an anode and the other electrode is a cathode; a DC voltage source capable of applying a constant DC voltage between the two electrodes, an insulator located in proximity of the two electrodes, and a gas filling a gap between the two electrodes, wherein cold plasma in the form of series of repetitive streamer breakdowns of the gas is generated, in response to the constant DC voltage applied between the two electrodes.

A method of producing and storing a sterilizing gas is disclosed. The method includes providing a flow of a gas into a chamber, generating cold plasma inside a chamber through repetitive streamer breakdowns of the gas in response to an applied DC voltage, resulting in the gas becoming a sterilizing gas, and containing the sterilizing gas in the chamber.

A method of sterilizing an object is disclosed. The method includes providing a flow of a gas into a chamber, generating cold plasma inside a chamber through repetitive streamer breakdowns of the gas in response to an applied DC voltage, resulting in the gas becoming a sterilizing gas, and exposing an object to be se sterilized to the sterilizing gas for a period of time.

### BRIEF DESCRIPTION OF DRAWINGS

Some of the figures shown herein may include dimensions. Further, some of the figures shown herein may have been created from scaled drawings or from photographs that are scalable. It is understood that such dimensions or the relative scaling within a figure are by way of example, and not to be construed as limiting. Objects and features of this disclosure will be better understood from the following description taken in conjunction with the drawing, wherein:

FIG. 1 is a schematic representation of the of the experiment setup used in experiments leading to this disclosure.

FIG. 2A shows current and voltage waveforms of series of the streamer breakdowns excited by the 5 kV DC voltage supplied to the electrode.

FIG. 2B shows current and voltage waveforms of an individual streamer breakdown. [t1]-[t6] are the time periods when a photograph of the streamer was taken.

FIG. 2C shows images of the streamer at certain time from [t1] to [t6]. The grounded electrode was located at d≈0.5 m. The He flow rate was 1 L/min.

FIG. 3 shows breakdown voltage required for triggering the first streamer for three different sizes of spherical electrodes. Threshold electric field required for the breakdown is about 2.5 kV/cm regardless the electrode size.

FIG. 4 shows streamer breakdowns vs. He flow rate for d=5 cm and U=5000 V.

FIG. 5 shows average period between streamer breakdowns vs. distance to the grounding plate d. Flow rate=1 L/min, U=5000 VDC.

FIG. 6 shows images of the DC voltage driven cold plasma device. (a) Length of the free jet is about 1 cm; (b) Length of the plasma jet can be extended to 2 cm if finger is placed nearby. He flow rate is 2 L/min and U=3400 V.

FIGS. 7A and 7B show current and voltage waveforms of the DC voltage driven cold plasma for multiple breakdown events and temporally resolved individual breakdown receptively.

FIG. 8 shows schematics of the DC voltage driven cold plasma device with hollow anode

FIG. 9A shows cross-sectional view of gas supply/HV electrode assembly in conventional geometry

FIG. 9B shows cross-sectional view of gas supply/HV electrode assembly in hollow anode configuration

FIG. 10A shows an image of the DC voltage driven cold plasma device of FIG. 8 being held in hand.

FIG. 10B is a demonstration of the cold nature of the plasma generated by the set up shown in FIG. 8.

FIG. 10C is a close-up view of the plasma generated by the set-up shown in FIG. 8.

FIG. 11A shows waveforms of current and voltage of series streamer breakdowns. Current is generated in pulses with a constant 5 kV applied voltage.

FIG. 11B shows waveforms of current, voltage, and Ne from a single streamer breakdown. [t1]-[t5] represents the times periods when the intensifier of the ICCD camera is working.

FIG. 11C shows photograph of the streamer at certain times from [t1] to [t5] taken in a single breakdown event.

FIG. 12 shows Tᵣₑₚ of the streamer breakdown vs. the applied voltage

FIG. 13 shows waveforms of current, Ne and ne from one streamer breakdown

FIG. 14 shows maximum of current and plasma density vs. applied voltage

FIG. 15A shows measured using optical emission spectroscopy and simulated spectrum when applied voltage was 5 kV

FIG. 15B shows Tᵣₒₜ and T_{vib} when applied voltage is between 4.7 kV and 5 kV.

FIG. 16 shows schematic drawing of the adaptable prototype of DC voltage driven cold plasma system. The system includes pressurized air tank, chamber for production of sterilizing gas with embedded multi-channel DC voltage driven cold plasma device and flexible hose with mass flow control to adjust flow of the sterilizing to specific application.

FIG. 17 is a schematic representation of one embodiment a cold-plasma generating system of this disclosure.

### DETAILED DESCRIPTION

For the purposes of promoting an understanding of the principles of the disclosure, reference will now be made to the embodiments illustrated in the drawings and specific language will be used to describe the same. It will nevertheless be understood that no limitation of the scope of the disclosure is thereby intended, such alterations and further modifications in the illustrated device, and such further applications of the principles of the disclosure as illustrated therein being contemplated as would normally occur to one skilled in the art to which the disclosure relates.

In this disclosure, a system generating cold plasmas in a self-oscillatory mode which is implying that constant in time driving voltage produces series of repetitive streamer breakdowns is described. Repetition frequency of the breakdowns is governed by the geometry of discharge electrodes/surroundings and gas flow rate. Each next streamer is initiated when the electric field on the anode tip recovers after the previous breakdown and reaches the breakdown threshold value. Repetition frequency of the streamer breakdowns excited using this principle can be simply tuned by reconfiguring the discharge electrode geometry. This custom-designed type of the cold plasmas, which operates on the DC high voltage and comprising of the series of the repetitive streamer breakdowns, is disclosed.

Cold plasmas of this disclosure driven by pure DC high voltage are free from the disadvantages AC-driven plasmas due to relief of requirements to the electrical insulation thickness, availability of large number of commercially offered power supplies and significant reduction of the EM radiation.

This disclosure cold plasma containing a series of repetitive streamer breakdowns which is excited by pure DC high voltage and demonstrates the ways to control the frequency of streamer repetition. A DC voltage driven cold plasma device operating on this principle and methods of practical use of the same are also disclosed.

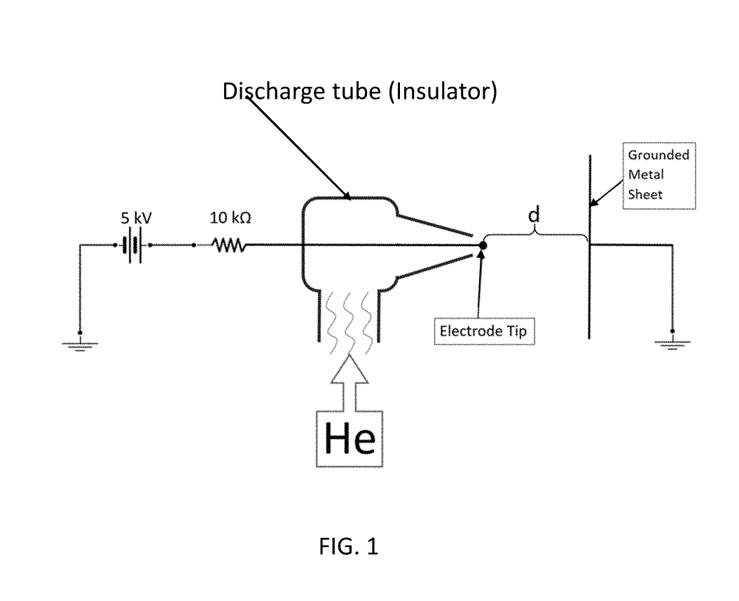

FIG. 1 shows schematic representation of the experimental setup used in experiments leading to this disclosure. Referring to FIG. 1, a DC power supply up to 5 kV was used in the experiments. A mass flow controller (Sierra SmartTrak 100) was used to control the helium supply into the exit point of the gas in the discharge tube. The high-voltage electrode on the axis of the discharge tube was immersed into the He which flowed through the discharge tube into open air. The discharge tube is made of an electrically insulating material such as glass. It should be noted that other insulating materials such as, but not limited to, plastic, Teflon, ceramics are possible to be used to make the discharge tube. The inner diameter of part of the discharge tube near where the He gas exits was 3.6 mm. A grounded metal sheet was installed outside the discharge tube at the distance d from where the He gas exits the discharge tube. The electrical current was measured using a 10 kΩ shunt resistor placed in series in the HV line as shown in FIG. 1.

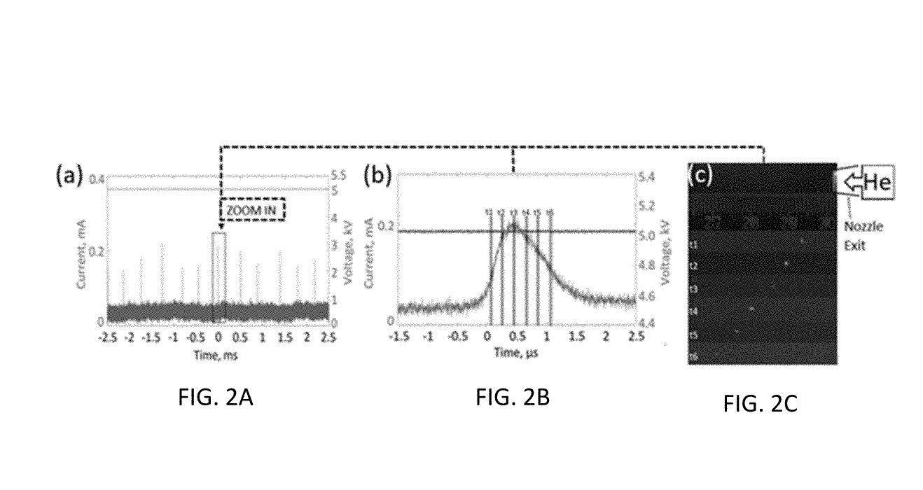

Series of streamer breakdowns were observed when 5 kV DC voltage was applied to the electrode in the system shown in FIG. 1 as shown in FIG. 2A. The time interval between two adjacent streamer breakdown pulses was 400-750 μs and amplitude of the current pulse was about 0.15-0.25 mA. For the set up shown in FIG. 1 to function to produce the cold plasma of this disclosure, it is important that the applied DC voltage stay constant for the duration of plasma generation. For purposes of this disclosure, constant DC voltage is intended to mean that variation of the applied DC voltage with time is less than 0.1%. A close look of the current and voltage waveforms of an individual discharge is shown in FIG. 2B. It is important to note that the applied DC voltage remains constant throughout the entire duration of the experiment including the period of streamer breakdown. In this setup the ground metal sheet was placed far away from exit point of the gas in order to eliminate its effect on the discharge behavior, whereas the surroundings were considered to provide the ground potential for discharge. Current pulse width was about 1.5 μs. The streamer development was photographed using an intensified charge-coupled device (ICCD) camera for the moments of time [t1]-[t6] indicated by the rectangular bars shown in FIG. 2B. Average velocity of streamer front propagation was ˜2.5·10⁶ cm/s.

Voltage was applied to the spherical electrode immersed in the helium flow using the setup shown in FIG. 1. The voltage was increased from zero up to the value Uₜₕ, when firing of the first streamer was detected (no space charge remaining from the previous breakdowns). In this case, the electric field (E) in vicinity of the spherical high voltage electrode is related to the tip voltage (U) as

$$E = \frac{U}{a} ,$$

where a is the radius of the electrode tip sphere. Thus, the threshold electric field (Eₜₕ) was determined as:

$${E}_{th} = \frac{{U}_{th}}{a} .$$

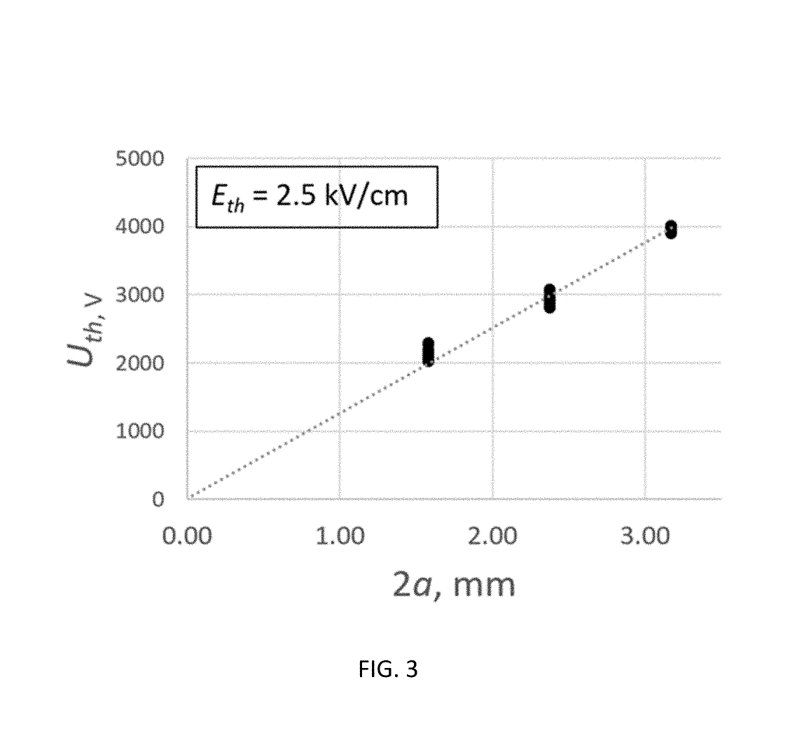

In order to evaluate critical electric field Eₜₕ required to fire the streamer, we used three spherical HV electrodes of diameters 1.59 mm, 2.38 mm and 3.18 mm. The dependence of Uₜₕ required to fire the first streamer on electrode tip diameter is shown in FIG. 3 (refers to the experimental set up shown in FIG. 1). One can see that the dependence was linear and crosses the origin. This clearly indicates that threshold electric field strength was constant, Eₜₕ=2.5 kV/cm, for all cases, which supports the operation mechanism formulated above.

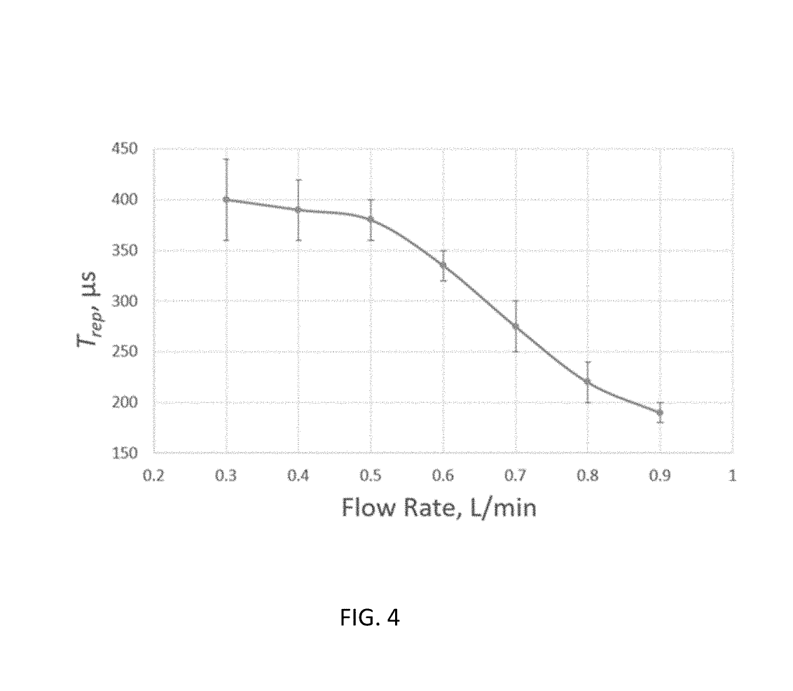

Next, the ways how frequency of repetition of streamer breakdowns can be controlled is considered. FIG. 4 shows the time interval between two adjacent streamer breakdowns (Tᵣₑₚ) vs. helium flow rate (refers to the experimental set up shown in FIG. 1). One can see that the time interval between breakdowns was reduced with the increase of He flow rate. This can be explained by the fact that larger He flow rate leads to faster He flow speed and this in turn results in faster removal of the positive space charge from the vicinity of electrode tip. Thus, electric field in vicinity of the streamer recovered faster to the threshold value Eₜₕ which led to more frequent pulsations. In addition, it was observed that more frequent streamer pulsations were more repeatable which is seen by decrease of the error bar for higher He flow rates.

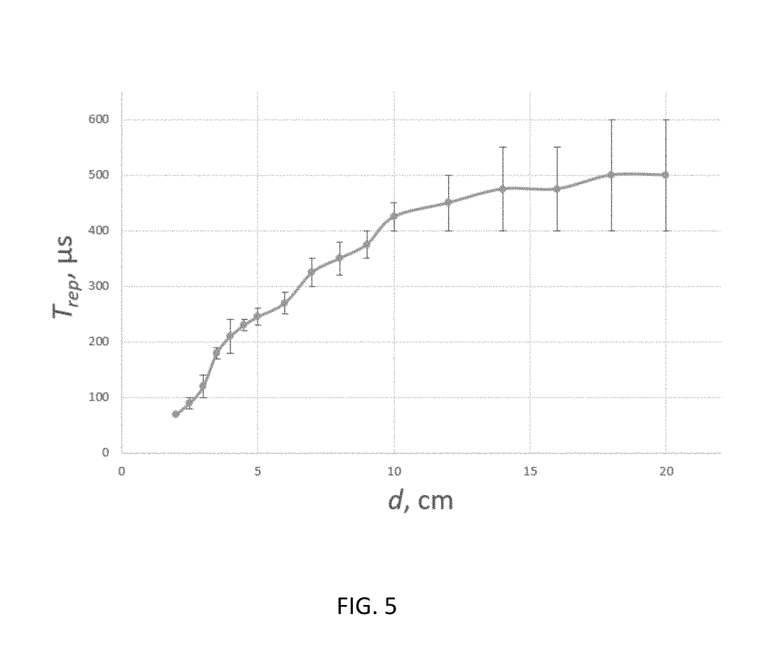

Another way to control the repetition time interval of streamer firing was to vary the background potential around the tip electrode. FIG. 5 is showing the relation between the repetition time interval and the distance d (refers to the experimental set up shown in FIG. 1). One can see from the plot that the decrease of the distance d caused more frequent streamer firings (period between the streamer firings Tᵣₑₚ decreased). More frequent streamer firings were associated with more stable and repeatable operation as indicated by the size of error bars in FIG. 5.

It should be noted that the experimental fact that Tᵣₑₚ increases for smaller d (FIG. 4) and for larger flow rate (FIG. 5) was obtained and confirmed multiple times in single experimental runs with real-time manual adjustment of distance d (or flow rate) in both directions and simultaneous observation of corresponding changes of Tᵣₑₚ on oscilloscope. However, spread of the measured Tᵣₑₚ between different experimental runs was quite high (up to 30%) due to high sensitivity of the source to precise geometry of grounded surroundings which is extremely hard to reproduce exactly the same in the different experimental runs. This spread can be traced by comparing of two Tᵣₑₚ measurements conducted at close conditions, but obtained in different experimental runs namely, Tᵣₑₚ=190 and 250 μs for d=5 cm and very close He flows 0.9 and 1 L/min.

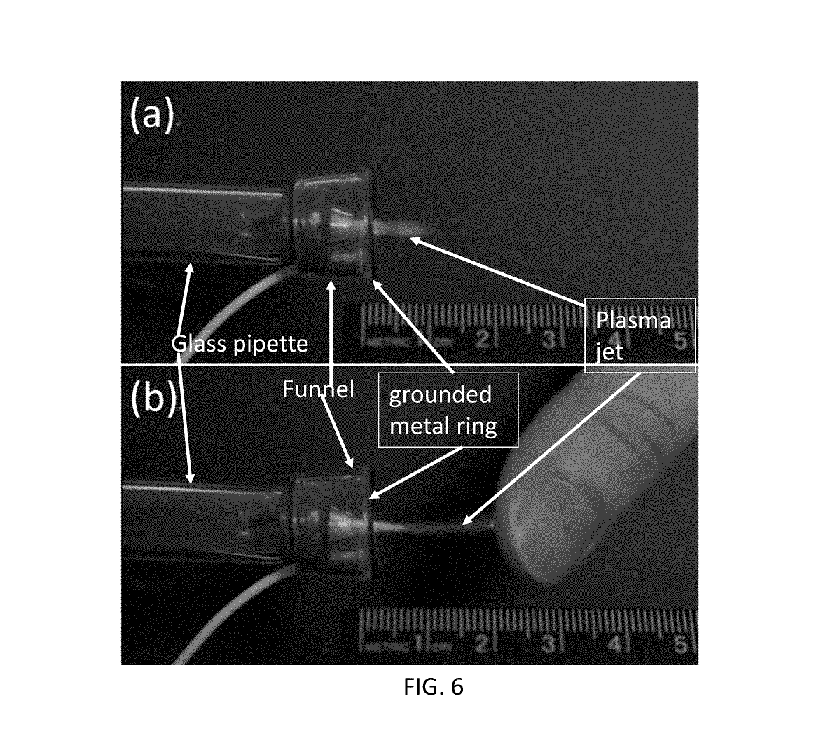

FIG. 6 is an image of another experimental set up used to create cold plasma according to the principles of this disclosure. Referring to FIG. 6, the set up uses a customized glass pipette and a funnel attached to it. The high voltage electrode is inserted into the tube near the discharge tube exit while grounded metal ring is fixed on the funnel. The grounded ring was used in order to vary background potential and thus to adjust the repetition frequency of the streamer breakdowns. Helium flow went through the center of the symmetric grounded ring. Several ring sizes and the distances from the pipette exit were tested in order to achieve steady and visible plasma as well as to extend the length of plasma. Within this range, the location of the grounded ring was determined in the design shown in FIG. 6 in order to fix the repetition frequency at around 15 kHz. The length of the free plasma jet was approximately 1 cm, while it can be extended to more than 2 cm if a finger is placed in vicinity as shown in part (b) of FIG. 6.

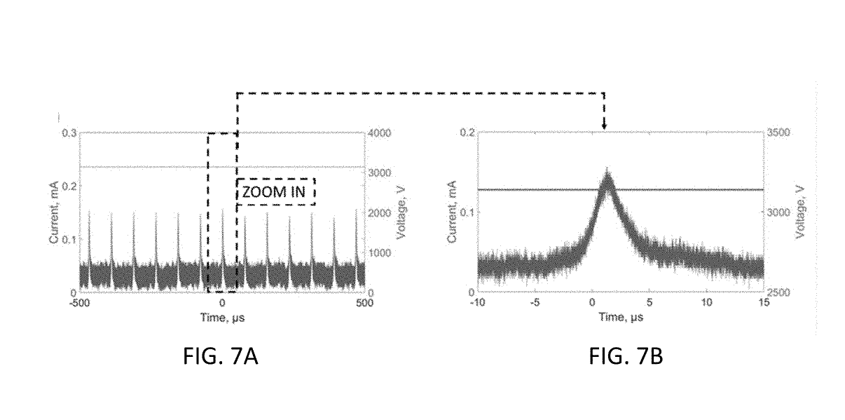

FIGS. 7A and 7B show voltage and current during the streamer breakdown (refers to experimental setup shown in FIG. 6). One can see that current amplitude was about 0.1 mA and repetition frequency—13 kHz. By comparing FIGS. 2A and 2B with FIGS. 7A and 7B respectively, one can see that the duration of the discharge increases by approximately factor of two. This can be explained by the closer proximity of the ground electrode for the case shown in FIGS. 7A and 7B that slows down the streamer front propagation and extend its temporal duration.

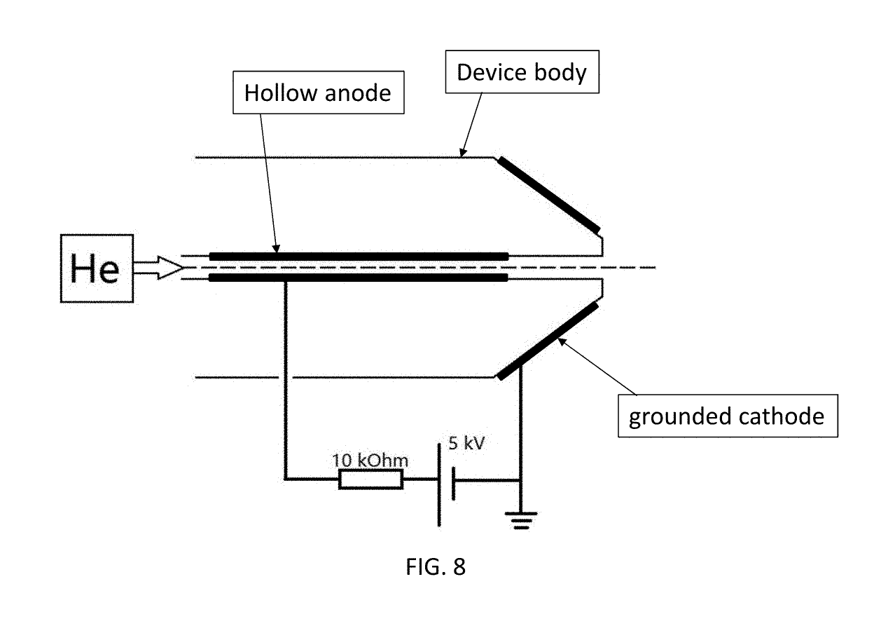

FIG. 8 shows is another DC-driven cold plasma device in accordance with the principles of this disclosure. This set up uses high voltage anode made of hollow metal tube with helium flow supplied through it. It is contains two electrodes: grounded electrode (cathode) and positive HV electrode (anode). The anode in this case is made of the hollow metal tube with gas (helium) being fed through that tube.

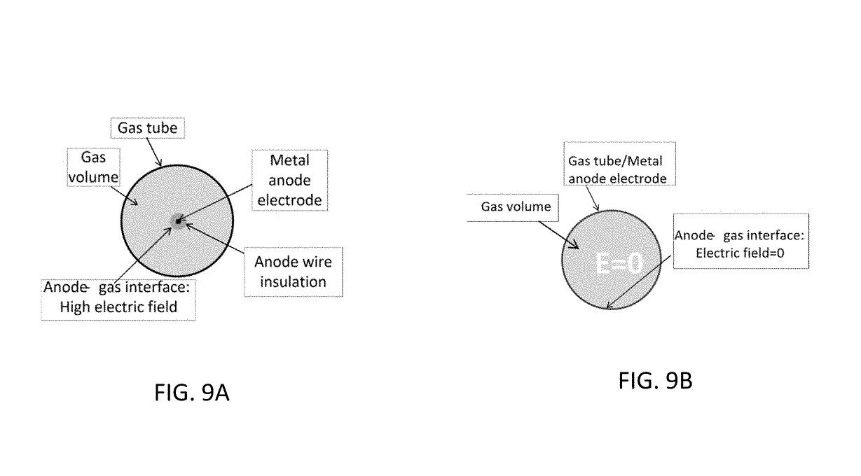

The set up shown in FIG. 8 differs from the arrangement shown in FIG. 1 and FIG. 6, where wire electrode is used as anode and that anode wire is fed through the larger diameter tube with gas flow as shown in FIG. 9A. For the case of conventional arrangement shown in FIG. 1 and FIG. 6, large electric fields are created on the anode-gas interface (or on the anode insulator—gas interface if insulated anode wire is used) as shown in FIG. 9B. This might cause ignition of the parasitic discharge around the anode along its entire length exposed to the gas flow. This parasitic discharge has adverse effect on the overall stability and parameter controllability of the plasma source.

The set up shown in FIG. 8 uses hollow anode when all gas flow is inside the anode. Since volume inside the metal anode tube is electrically equipotential, electric field on the anode-gas interface is equal to zero (see FIG. 9B). This prevents ignition of the parasitic discharge around the anode-gas interface and restricts the discharge location only to the edge of the anode tube where it is desired. This has positive impact on stability of the source and controllability of plasma parameters.

Specific sizes of the components of the device shown on FIG. 8 are given below. It should be noted that these sizes are not intended to be limiting in any fashion. The main body of the DC-driven plasma device of FIG. 8 is made from a Teflon cylinder with diameter of 1″ and height of 5.5″. The end surface of the device has a flat area with diameter of 0.3″ then a conical cut is made until the OD of the Teflon. The cone is wrapped with copper foil which is grounded. The center hole has a diameter of ⅛″ where helium gas is fed. Inside of the hole lies the HV electrode. It is a copper tube inserted onto the wall of the hole where helium flows through. The tube is hidden inside of the device by a distance of about 0.55″ measured from the exit.

DC voltage driven cold plasma device shown schematically in FIG. 8 uses a 10 kΩ resistor is connected in series on the HV line. The device utilizes a coaxial cable connected to a DC HV power supply. The power supply used for testing is Bertan 225-05. It also utilizes a gas line to supply helium. The helium is controlled by a mass flow controller (Alicat MC-10SLPM).

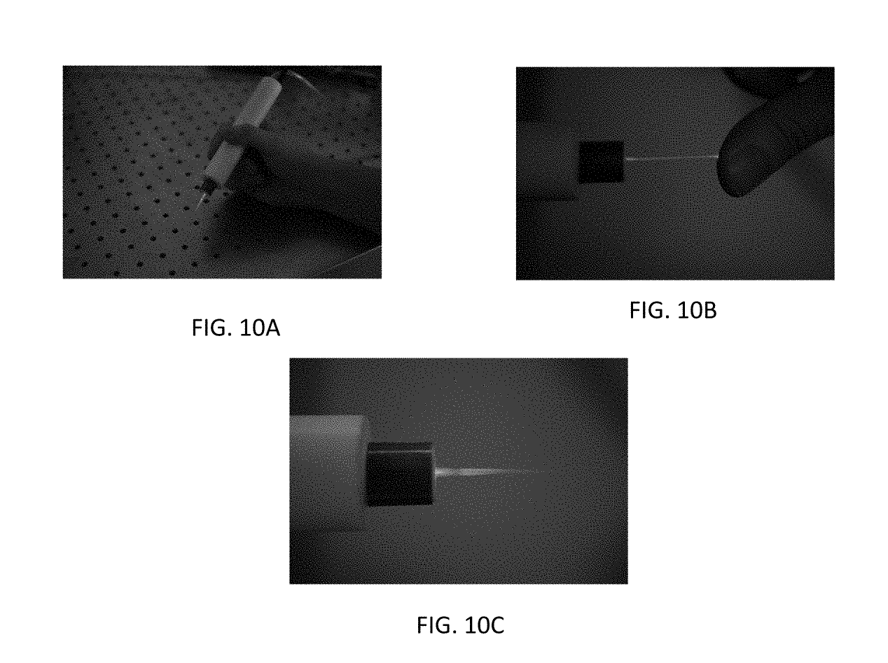

FIGS. 10A, 10B and 10C show some images of the DC voltage driven cold plasma device shown on FIG. 8.

FIG. 10 A is an image of the device build held in hand. FIG. 10B is a demonstration of the cold nature of the plasma that it can be touched by a human hand. FIG. 10 is a close-up view of the plasma generated by the set up shown in FIG. 8. This assembly generates plasma jets with a length of up to 3 cm and operates in self-oscillatory mode with current bursts repetition frequency of around 3 kHz. The helium flow rate during the experiment was chosen to be 5 LPM. Operation of the source at lower flow rates (<3 LPM) can be used if arcing between the electrodes is required. Operation at higher flow rates (>6 LPM) can be used to obtain turbulent jet.

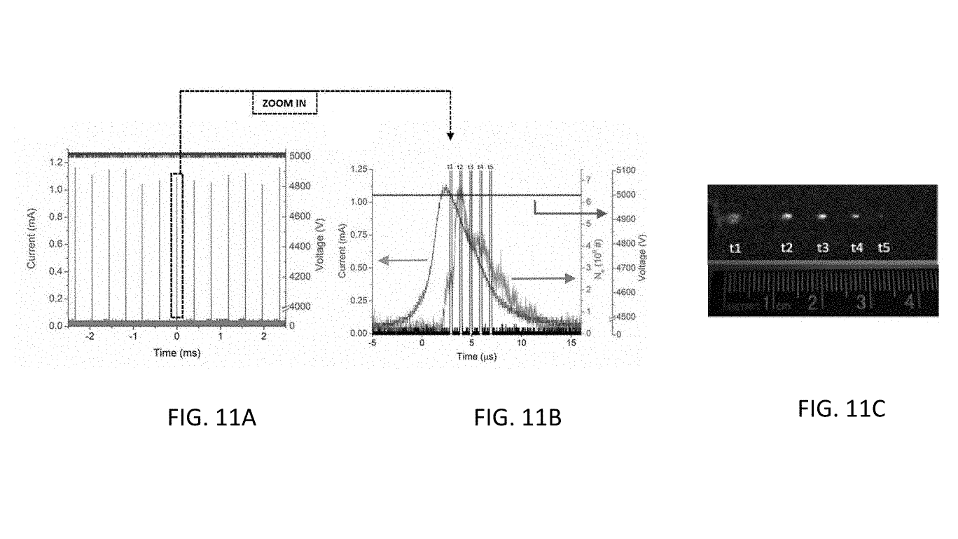

FIGS. 11A through 11C show several characteristics of the device shown in FIG. 8 FIG. 11A shows the temporal evolution of discharge voltage and discharge current. FIG. 11B shows, total electron number in plasma jet Nₑ and close-up view of discharge current for selected event in FIG. 11A. FIG. 11C shows an image of streamer propagation. One can see from that sequence of the breakdowns at a frequency of about 2.75 kHz was generated at application of 5 kV DC voltage and each current pulse had an amplitude of about 1.0-1.2 mA. A close look of the voltage, current and total number of electrons Nₑ of an individual breakdown is shown in FIG. 11B. It can be observed that the current pulse duration was about 5 μs and the voltage was constant at 5 kV during the breakdown. The peak value of Nₑ was about 6.5*10⁹ and the rise of Nₑ was delayed by approximately 2 μs from the moment of discharge current maximum. The propagation of the streamer captured in a single breakdown event using ICCD camera (Princeton Instruments PI-MAX4) is shown in FIG. 11C. The rectangles in FIG. 11B, t1-t5, represent the moments at which images shown in FIG. 11C were taken. The multiple locations of a singular streamer tip are also labeled in FIG. 11c respectively. The average velocity of the streamer propagation was estimated to be around 6.67*10⁵ cm/sec. Comparing with the embodiment shown in FIG. 6, the visible length of the jet was extended by three times, the peak value of current increased by about ten times, duration of the pulses also was slightly expanded, and the repetition frequency decreases from 15 kHz to 2.75 kHz.

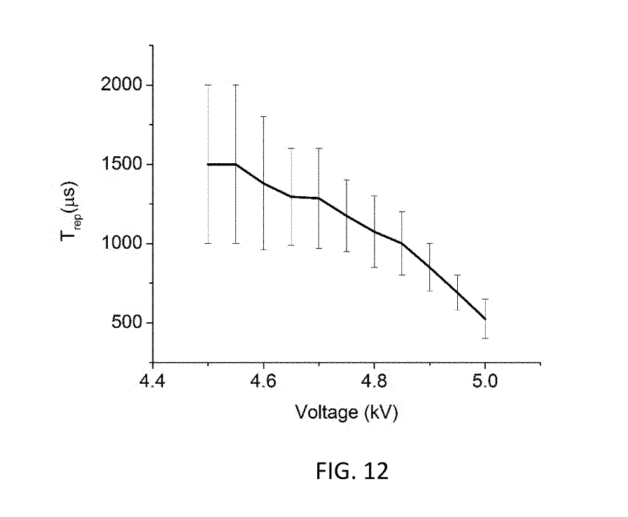

The correlation between the time between the breakdowns Tᵣₑₚ and the applied voltage is shown in FIG. 12 (refers to the set up shown in FIG. 8). One can see that, as voltage increases, Tᵣₑₚ decreases. Tᵣₑₚ decreased from 1500 μs to 500 μs with voltage increase from 4.5 to 5 kV, which can be explained by faster recovery of the electric field on the HV electrode to the breakdown threshold value when the electrode carried higher potential. In addition, vertical error bars indicate larger variability of the time between the breakdown events at lower voltages.

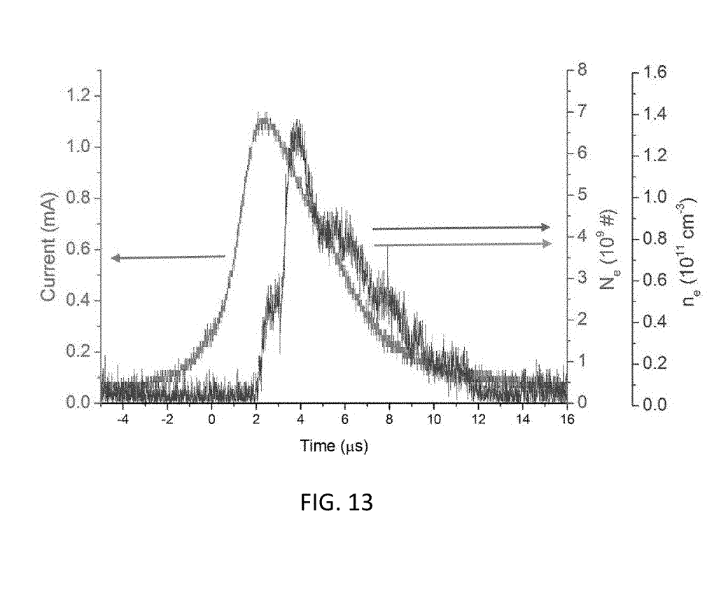

FIG. 13 shows the evolution of nₑ with respect to time along with the change of current and Ne (referring to the device shown in FIG. 8). Average plasma density nₑ in the plasma jet was determined as nₑ=Nₑ/V₀ using constant plasma volume V₀=0.05 cm³ (cylinder with 0.2 cm diameter and 1.5 cm height). One can see from FIG. 13 that, the trend of nₑ overlaps with the trend of Nₑ, reaching a maximum value of 1.4*10¹¹ cm ⁻³ about 2 μs after current reaches its peak.

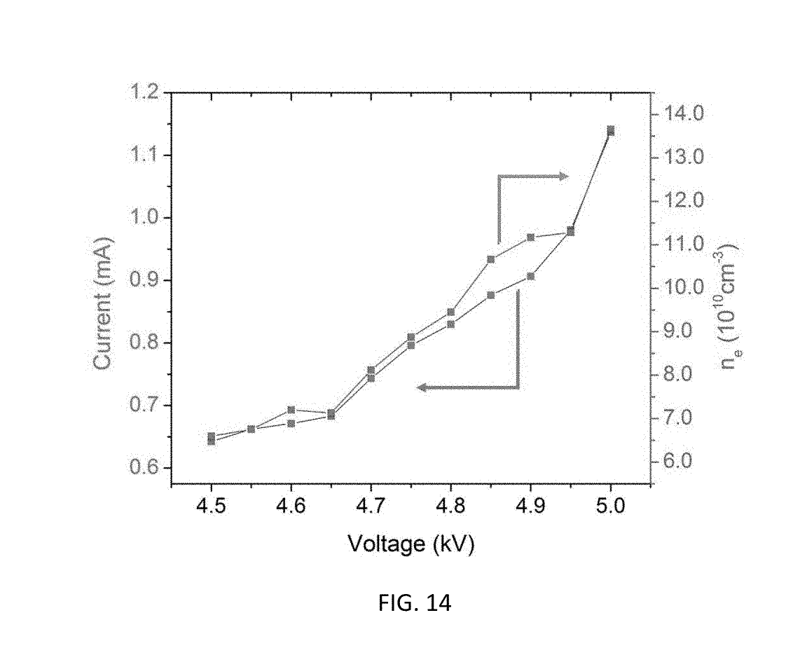

FIG. 14 shows how plasma density nₑ changes with the applied voltage (refers to the set up shown in FIG. 8). The maximum values of current and nₑ in a single pulse breakdown with respect to the applied voltage are shown in FIG. 14 when varying the voltage from 4.5 kV to 5 kV. One can see that both the peak current and peak ne increase with the applied voltage, and they follow a similar trend. Peak current increases from 0.65 mA to 1.15 mA and maximum of nₑ increases from 1.7*10¹¹ cm⁻³ to 3.5*10¹¹ cm⁻³.

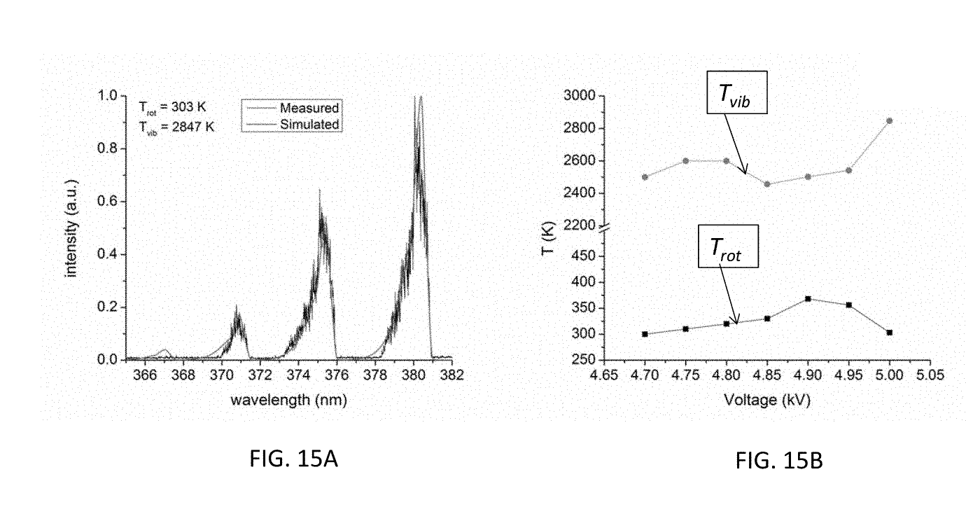

FIG. 15A shows measured radiation spectrum in the range 360-380 nm for the applied voltage of 5 kV, which corresponds to the second positive system of nitrogen (refers to the device shown in FIG. 8). Also best spectra fitting results using SPECAIR software are shown. The resulted T_{vib} was 2847 K and Tᵣₒₜ was 303 K. FIG. 15B shows dependence of T_{vib} and Tᵣₒₜ on the applied voltage. After running SPECAIR for the spectrum at applied voltages ranging from 4.7 kV to 5 kV, it was found that there was no strong dependence of T_{vib} and Tᵣₒₜ on the applied voltage. T_{vib} was in the range of 2675±175 K and Tᵣₒₜ was in the range of 338±38 K.

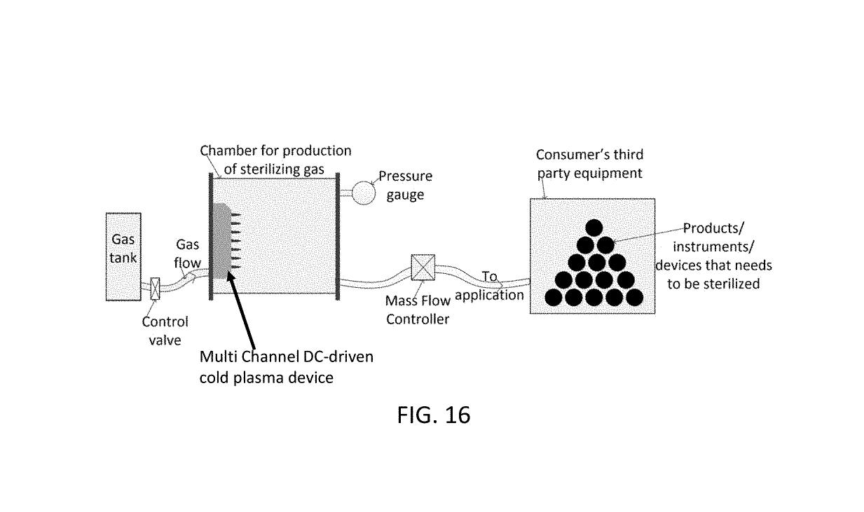

FIG. 16 is a system demonstrating how devices of this disclosure can be exploited in practical applications. Referring to FIG. 16, scaled-up multi-channel DC voltage driven cold plasma device having diameter of about 10-15 cm that incorporates about 100 individual channels for gas feeding (e.g. air). Each individual channel creates single plasma breakdown and enriches air passing through this channel with sterilizing reactive species. Particular values of voltage can be 20 kV and overall discharge current of about 100 mA (about 1 mA per individual channel) but not limited to these values only.

System shown in FIG. 16 also includes a chamber (e.g. cylinder of 15-20 cm in diameter and 60-80 cm in length—approximately 30 liters). Multi-channel DC voltage driven cold plasma device is embedded in one of the sidewalls of the chamber. Pressure inside the chamber is monitored using gas independent Baratron pressure gauge. Typically the chamber used to contain the sterilizing gas is sealed from contact with ambient atmospheres.

System shown in FIG. 16 operates as follows. Air is fed from the pressurized tank into the multi-channel DC voltage driven cold plasma device embedded into the chamber for production of sterilizing gas using flexible hose. Sterilizing gas from the chamber is delivered to the consumer using flexible hose. Mass Flow Controller (MFC) is used in the outlet hose from the chamber in order to set flow rates to the range required by each specific consumer. Generally, consumer of the sterilizing gas refers to the specific third-party system or equipment loaded with products/tools/instruments/devices that require sterilization. After that input valve supplying compressed air into the chamber is adjusted using pressure gauge and controller so that pressure in the chamber is slightly exceeds the atmospheric pressure (positive pressure chamber). This prevents penetration of outside air inside the gas flow and ensures same conditions for the operation of the cold plasma device regardless the specific flow rate required by specific consumer. The system throughput of the sterilizing gas is up to 20 LPM.

Application of this invention can be explained on the example of the embodiment shown in FIG. 16. This system is readily compatible with maximal variety of third-party equipment and does not require any changes into that equipment. Indeed, our device is not incorporated into the unique third-party equipment used by the consumer. Instead, our system shown in FIG. 16 is used only for production and temporary storage of the sterilizing gas. Shortly after the creation, the sterilizing gas is fed through a flexible hose connected to the specific third-party consumer equipment which is loaded with products/tools/instruments/devices that require sterilization. In many cases, the sterilizing gas outlet hose from our system will simply connect to the inlet line of the conventional sterilizing agent (e.g. hydrogen peroxide) in the specific third-party equipment used by the consumer. Examples of consumers' products/tools/instruments/devices that require sterilization include perishable fresh produce, perishable foods, dried foods, medical instruments, tools and devices etc.

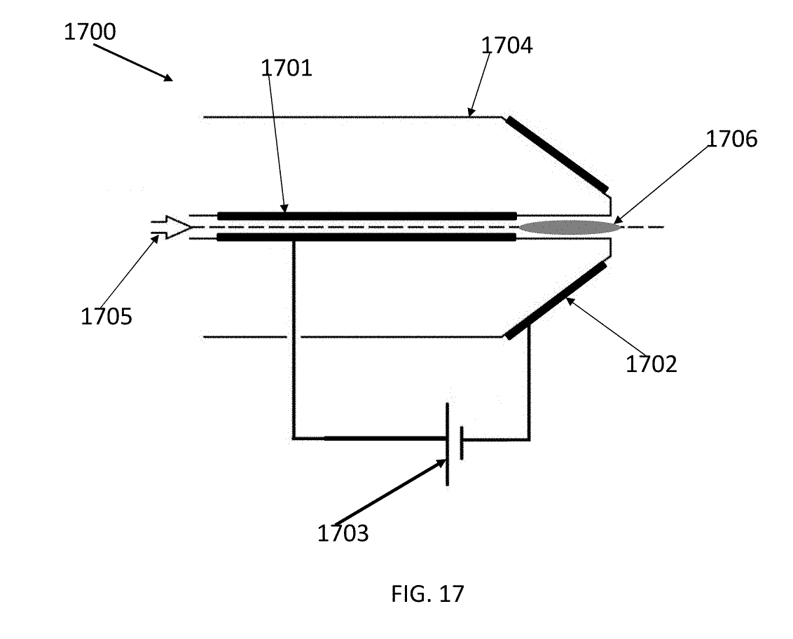

Based on the above description, FIG. 17 is a schematic representation of one embodiment of a cold-plasma generating system 1700 of this disclosure. Referring to FIG. 17, electrodes 1701 and 1702 are two electrodes. In this schematic, 1701 is shown to be a hollow electrode and 1702 is shown to be a ring electrode. However, there is no limitation that the electrodes should be hollow or ring-shaped. One of them can be an anode while the other is a cathode. Referring again to FIG. 17, 1703 is a DC voltage source capable of supplying a DC voltage between the two electrodes. An insulator 1704 is located either in contact or in close proximity to the two electrodes as shown in FIG. 17. For purposes of this disclosure, close proximity is intended to mean a distance in the same order of magnitude as the maximum distance between the two electrodes. Referring again to FIG. 17, under the action of applied DC voltage form the DC voltage source 1704, gas 1705 undergoes repetitive streamer breakdowns generating a cold plasma.

While above embodiments are described above operating with just one specific type of gas this invention is not restricted to that only. The invention can operate with variety gases including air, nitrogen, oxygen, inert gases etc.

It should be noted that insulator should be located in vicinity of the electrodes. The material of the insulator can be Teflon, ceramics etc. The shape of the insulator can be different such as flat plate, hollow tube, rod etc.

It should be noted that geometric shape of anode and cathode electrodes can be different. They can be pin electrodes, hollow electrodes, single wire, flat plate, mesh etc.

The cold plasma created by the invented device can be in a different shapes including jet, filament, multiple of filaments, spark, multiple sparks etc.

It should be noted that geometric shape of the plasma is not limited to jet only. The cold plasma created by the invented device can be in a different shapes including jet, filament, multiple of filaments, spark, multiple sparks etc.

It should be noted that the methods and systems of this approach use constant voltage applied between the discharge electrodes throughout the system operational time, thereby eliminating problems with EMI, leakage currents and safety concerns.

This invention creates market opportunity on the market of freeze-drying (lyophilization) equipment. Lyophilization is a process of removing moisture from products (foods, pharmaceuticals etc.) for a preservation and prolonged storage of perishables. Sterilization is an important step of the lyophilization process, which is achieved using inefficient and expensive steam or vapor hydro peroxide (VHP) systems currently. Lyophilization Equipment and Services market was valued $15.9 billion in 2012 (developing at a large CAGR 10.4%) and $28.7 billion is forecasted for 2018. Current lyophilization technology uses steam or vapor hydro peroxide (VHP) for sterilization prior to the main freeze-drying cycle, which suffers from multiple disadvantages. This includes long duration of the sterilization cycle (sterilization takes up to 4 hours of operation and can be up to 20% of the total lyophilization cycle), residual chemicals remaining on the products, damage to lyophilization chamber due to use of very strong oxidizing agent, necessity to perform a pre-surface-drying of the products and overall system complexity and cost. DC voltage driven cold plasma technology will be able to lower the cost of the sterilization system, to enable in-situ sterilization during freeze-drying cycle which can save up to 20% of total cycle time, to operate at room temperature, to eliminate strict requirements for compatibility of freeze-dryer chamber with strong oxidizing agent, and to achieve higher overall simplicity of the sterilization system and procedure. Therefore, Total Addressable Market of Lyophilization Equipment and Services is a large-size multi-billion dollar opportunity. We target segment of that market concerned with manufacturing and servicing sterilization accessories for freeze-dryers. Tremendous advantages of the DC voltage driven cold plasma technology over traditional sterilization approach can propose attractive alternative for final customers, so that cold plasma technology has great potential to seize that market segment.

Other large market opportunities for our invention include packaged food and medical devices markets. The global market size for the packaged food is $2.35 trillion in 2014 and is predicted to reach $3.03 trillion by the year of 2020 with CAGR of 4.5%. Besides, the market size for the food packaging industry is $305.96 billion by the year of 2019 globally. It includes the packaging for the frozen food, which is worth $54.53 billion by the year of 2014 with an estimated CAGR of 4.87%; as well as the fresh food packaging which has a market size of $95.91 billion by 2020 and has a CAGR of 3.38% between 2015-2020. Another potential market would be the packaging for medical devices. It is projected to worth $21.64 billion with a 5.9% increase in 2016 and is projected to reach $30.5 billion by 2021 globally.

Based on the above description, it is an objective of tis disclosure to describe a plasma generating system capable of generating a cold plasma. The plasma generating system capable of generating a cold plasma includes two electrodes, wherein one of the two electrodes is an anode and the other electrode is a cathode; a DC voltage source capable of applying a constant DC voltage between the two electrodes; an insulator located in proximity of the two electrodes; a gas filling a gap between the two electrodes, wherein cold plasma in the form of series of repetitive streamer breakdowns of the gas is generated, in response to the constant DC voltage applied between the two electrodes. For purposes of the description of the plasma generating systems of this disclosure, proximity of the insulator is defined in connection with scale of the system. For the embodiments described above distance of between the electrodes was from few millimeters to about several centimeters. Accordingly, insulator located in proximity refers to distances electrode-insulator was below several centimeters. It has to be noted, that if the entire system invented here is scaled up the electrode insulator distance can be also scaled up. Generally, distances between the electrodes are in the range from millimeter to several meters, distances between electrode and insulator can vary the zero to several meters.

The two electrodes in the plasma generating system of this disclosure can be made of a metal or an alloy. A non-limiting example of a metal that can be used for this purpose is copper while a non-limiting example of an alloy that can be used for this purpose is an alloy of copper. A non-limiting example of an insulator that can be used in the plasma generating system of this disclosure is Teflon (Registered Tarde Mark). The insulator can be a hollow tube or flat plate or other shape.

A non-limiting range for the constant high-voltage in the system of this disclosure is 1000-500,000 volts. Non-limiting examples of gases that can be used in the plasma-generating system of this disclosure include air, nitrogen and helium.

It should be recognized that in some embodiments of the plasma generating system of this disclosure, the two electrodes are hollow. Non-limiting examples for the shape of the electrodes include flat plate and mesh. It should be noted that the shape of the electrodes can be varied and taken in different combinations. For example, hollow cathode and ring anode, pin cathode and wire anode, pin cathode and ring anode etc.

It is another objective of this disclosure to describe a method of producing and storing a sterilizing gas The method includes providing a flow of a gas into a chamber, generating cold plasma inside the chamber through repetitive streamer breakdowns of the gas in response to an applied DC voltage, resulting in the gas becoming a sterilizing gas; and containing the sterilizing gas in the chamber. Gases suitable for this method include, but not limited to air, nitrogen, and helium. In one embodiment of the method of producing and storing a sterilizing gas includes additional steps of transferring the sterilizing gas into another chamber and transporting it. FIG. 16 illustrates one example of such a transfer and/or transport.

It is another objective of this disclosure to describe a method of sterilizing an object. The method includes providing a flow of a gas into a chamber, generating cold plasma inside a chamber through repetitive streamer breakdowns of the gas in response to an applied DC voltage, resulting in the gas becoming a sterilizing gas, and exposing an object to be se sterilized to the sterilizing gas for a period of time. The period of time depends on the degree of sterilization required. In some cases, the degree of sterilization required may be determined through microscopic observations of bacteria or other qualitative and quantitative analytical methods of analysis and observation using sophisticated chemical and bacterial analysis instrumentations and methods. Non-limiting examples of objects that can be sterilized using the method of this disclosure include, but not limited to surgical instruments and produce. Example so produce that can be sterilized include, but not limited to plants, vegetables, fruits and leafy vegetables.

While the present disclosure has been described with reference to certain embodiments, it will be apparent to those of ordinary skill in the art that other embodiments and implementations are possible that are within the scope of the present disclosure without departing from the spirit and scope of the present disclosure. Thus, the implementations should not be limited to the particular limitations described. Other implementations may be possible. It is therefore intended that the foregoing detailed description be regarded as illustrative rather than limiting. Thus, this disclosure is limited only by the following claims.

1. A plasma generating system capable of generating a cold plasma comprising:
two electrodes, wherein one of the two electrodes is an anode and the other electrode is a cathode;
a DC voltage source capable of applying a constant DC voltage between the two electrodes;
an insulator located in proximity of the two electrodes; and
a gas filling a gap between the two electrodes;
wherein cold plasma in the form of series of repetitive streamer breakdowns of the gas is generated, in response to the constant DC voltage applied between the two electrodes.

2. The system of claim 1, wherein the anode is a metal or an alloy.

3. The system of claim 2, wherein the metal or alloy is copper.

4. The system of claim 1, wherein the cathode is a metal or an alloy.

5. The system of claim 4, wherein the metal is copper.

6. The system of claim 4, wherein the alloy is an alloy of copper.

7. The system of claim 1, insulator is Teflon.

8. The system of claim 1, wherein the high voltage is in the range of 1000-500,000 volts.

9. The system of claim 1, wherein the gas is one of air, nitrogen or helium.

10. The system of claim 1, where one electrode is hollow and other is ring shaped.

11. The system of claim 1, wherein the electrode is pin shaped and other is mesh shaped.

12. The system of claim 1, wherein the two electrodes are in the shape of a mesh.

13. The system of claim 1, wherein the insulator is a hollow tube.

14. The system of claim 1, wherein the insulator is in the shape of a flat plate.

15. A method of producing and storing a sterilizing gas, comprising:
providing a flow of a gas into a chamber;
generating cold plasma inside a chamber through repetitive streamer breakdowns of the gas in response to an applied DC voltage, resulting in the gas becoming a sterilizing gas; and
containing the sterilizing gas in the chamber.

16. The method of claim 15, wherein the gas is one of air, nitrogen, and helium.

17. The method of claim 15, further comprising transferring and transporting the sterilizing gas to a point of use.

18. A method of sterilizing an object comprising:
providing a flow of a gas into a chamber;
generating cold plasma inside a chamber through repetitive streamer breakdowns of the gas in response to an applied DC voltage, resulting in the gas becoming a sterilizing gas; and
exposing an object to be se sterilized to the sterilizing gas for a period of time.

19. The method of claim 18, where in the object to be sterilized is a surgical instrument.

20. The method of claim 18, wherein the object to be sterilized is one of a vegetable, a fruit, a leafy vegetable, and a plant.
